(19) United States
(12) Patent Application Publication
(10) Pub. No.: US 2026/0057587 A1
(43) Pub. Date: Feb. 26, 2026

(54) FAST MOTION-RESOLVED MRI RECONSTRUCTION USING SPACE-TIME-COIL CONVOLUTIONAL NETWORKS WITHOUT K-SPACE DATA CONSISTENCY

(71) Applicants: Memorial Sloan-Kettering Cancer Center, New York, NY (US); Memorial Hospital for Cancer and Allied Diseases, New York, NY (US); Sloan-Kettering Institute for Cancer Research, New York, NY (US)

(72) Inventors: Ricardo Otazo, New York, NY (US); Victor Murray, New York, NY (US); Anthony Mekhanik, New York, NY (US); Ramin Jafari, New York, NY (US)

(21) Appl. No.: 19/111,268

(22) PCT Filed: Sep. 12, 2023
(86) PCT No.: PCT/US2023/073926

§ 371 Date: Mar. 12, 2025

Related U.S. Application Data
(60) Provisional application No. 63/406,053, filed on Sep. 13, 2022.

(51) Int. Cl. G06T 11/00; G06N 3/0464

(52) U.S. Cl. CPC G06T 11/006; G06N 3/0464

(57) ABSTRACT

Systems and methods for fast reconstruction of motion-resolved magnetic resonance images using space-time-coil convolutional networks are disclosed. The system can receive a plurality of k-space data sets. The system can detect a motion signal therefrom. The system can classify the k-space data sets according to states of the motion signals. The system can resolve the k-space data set to Euclidean space images. The system can resolve the Euclidean space images to a combined Euclidian space image. For example, the system can use a convolutional network that exploits spatial, temporal and coil correlations without k-space data consistency to minimize computation time.

### CROSS-REFERENCE TO RELATED PATENT APPLICATIONS

This application claims the benefit of and priority to U.S. Provisional Patent Application No. 63/406,053, filed Sep. 13, 2022, including the specification, drawings, claims and abstract, is incorporated herein by reference in its entirety.

### STATEMENT OF U.S. GOVERNMENT SUPPORT

This invention was made with government support under CA255661, awarded by the National Institutes of Health. The government has certain rights in the invention.

### TECHNICAL FIELD

The present application relates generally to the formation of dynamic four-dimensional images.

### BACKGROUND

Some medical imaging techniques, such as magnetic resonance imaging (MRI), can include dynamic images of certain type of physiological motion taken over a period of time. Physiological motion can include respiration, heartbeats, and other involuntary or unavoidable movements, at least under some circumstances. These images can include four dimensions, including the spatial dimensions and the motion dimension. Moreover, the images can be taken with respect to a plurality of coils, which may differ based on the position thereof, and the tissue the coil originated or destined signal transits through. The image data can be captured in k-space domain. The use of golden-angle radial trajectories, where two consecutive radial lines are separated by the golden angle, can be employed to continuously acquire the data for multiple motion cycles and to retrospectively sort the acquired data into motion states to form the fourth dimension. Four dimensional (4D) MRI with high spatial and temporal resolution benefits from (1) fast acquisition to have a clinically feasible scan time and (2) fast reconstruction to deal with the large amount of data to be processed. Fast acquisition can be accomplished by reducing the number of k-space points, at the expense of using an iterative reconstruction algorithm that exploits image compressibility and different coil sensitivities subject to data consistency in k-space. Iterative reconstruction can be particularly slow for 4D MRI since data consistency has to be enforced in each time point and coil element for each iteration requiring two three dimensional (3D) Fourier transforms per time point and coil element.

### SUMMARY OF THE INVENTION

The systems and methods of the present disclosure provide techniques for collecting continuous k-space data in the presence of motion and reconstructing 4D images, where the fourth dimension represents motion states, using a convolutional neural network that exploits spatial, temporal and coil correlations without the need to enforce explicit data consistency in k-space. For example, k-space data can be continuously accumulated according to a series of radial lines, where each line traverses the center of k-space. A motion signal, such as a cardiac or respiratory cycle can be detected according to the k-space data. For example, the center of k-space represents the average of all pixels in the image domain and can be used as a surrogate for motion. The k-space data can be classified according to the position of each k-space point in the motion signal. For example, the amplitude of the motion signal can be divided into bins and the absolute value of the central k-space point in each radial can be used to assign a motion bin or state to each radial line. After sorting the acquired k-space data into motion states, an inverse Fourier transform can be applied to generate images in each motion state and coil resulting in a five-dimensional (5D) image. The 5D image may have aliasing artifacts since the scan time will be limited and there is not enough data to reconstruct images with full information. A convolutional neural network will receive the aliased 5D image and generate at least one image per state of the motion signal and combine all the coils. The convolutional neural network can evaluate portions of associated images, such as at other motion states, from other coils, or in other planes to resolve an image comprising information available in a plurality of the received images. For example, the convolutional neural network can resolve a higher quality image (e.g., additional detail, higher contrast, etc.) from a plurality of received images. Therefore, the systems and methods described herein can remove artifacts associated with limited k-space data to resolve physiologic motion as a new dimension. Moreover, the convolutional neural network can operate entirely in the image domain without checking data consistency in k-space to significantly reduce computation time compared to iterative reconstruction techniques (e.g., for 4D image reconstruction in real-time).

At least one aspect of the present disclosure is directed to a system for dynamic image reconstruction. The system can include one or more processors coupled to a non-transitory memory. The system can receive a plurality of k-space datasets. The system can detect a motion signal from the plurality of k-space datasets. The system can classify each of the plurality of k-space data sets according to a state of the motion signal. The system can resolve the plurality of the k-space datasets to a plurality of first images, each of the plurality of first Euclidean space images corresponding to one of the plurality of k-space datasets. The system can convey the plurality of first Euclidean space images and corresponding image acquisition data to an image reconstruction network. The system can resolve, by the image reconstruction network, a second Euclidean space image, based on the plurality of first Euclidean space images without checking data consistency in k-space.

In some implementations, the k-space data sets are radially sampled. In some implementations, the k-space data sets are generated for each of a plurality of coils of the MRI machine. In some implementations, the motion signal corresponds to a cardiac cycle or a respiratory cycle. In some implementations, the states of the motion signal are defined according to an amplitude of the motion signal. In some implementations, the k-space data sets are three dimensional. In some implementations, the image reconstruction network is a residual U-net network. In some implementations, the image reconstruction network employs patch mixing between at least Euclidean, coil, and motion signal state axes.

At least one aspect of the present disclosure relates to a method for image reconstruction. The method can be performed, for example, by a data processing system. The method includes receiving a plurality of k-space data sets. The method includes detecting a motion signal from the plurality of k-space. The method includes classifying each of the plurality of k-space data sets according to a state of the motion signal. The method includes resolving the plurality of the k-space data sets to a plurality of first images, each of the plurality of first images corresponding to one of the plurality of k-space data sets. The method includes conveying the plurality of first images and corresponding image acquisition data to an image reconstruction network of the data processing system. The method includes resolving, by an image reconstruction network, a second image, based on the plurality of first images.

In some implementations, the k-space data sets are radially sampled. In some implementations, the k-space data sets are generated for each of a plurality of coils of the MRI machine. In some implementations, the k-space data sets are three dimensional. In some implementations, the motion signal is one-dimensional. In some implementations, the motion signal corresponds to a cardiac cycle or a respiratory cycle. In some implementations, the states of the motion signal are defined according to an amplitude of the motion signal. In some implementations, image reconstruction network is a residual U-net network. In some implementations, the image reconstruction network employs patch mixing between at least a plurality of Euclidean axes, a coil axis, and a motion signal state axis.

At least one aspect of the present disclosure relates to a computer-readable medium storing instructions that, when executed by a one or more processors, cause it to perform a process. The process can include receiving a plurality of k-space data sets. The process can include detecting a motion signal from the plurality of k-space data sets. The process can include classifying each of the plurality of k-space data sets according to a state of the motion signal. The process can include resolving the plurality of the k-space data sets to a plurality of first images, each of the plurality of first images corresponding to one of the plurality of k-space data sets. The process can include conveying the plurality of first images and corresponding image acquisition data to an image reconstruction network of the data processing system. The process can include resolving a second image, based on the plurality of first images.

In some implementations, the k-space data sets are received from each of a plurality of coils of an MRI machine. In some implementations, the k-space data sets are three dimensional. In some implementations, the motion signal is one-dimensional. In some implementations, the motion signal corresponds to a cardiac cycle or a respiratory cycle. In some implementations, the states of the motion signal are defined according to an amplitude of the motion signal. In some implementations, the image reconstruction network is a residual U-net network. In some implementations, the image reconstruction network employs patch mixing between at least a plurality of Euclidean axes, a coil axis, and a motion signal state axis.

These and other aspects and implementations are discussed in detail below. The foregoing information and the following detailed description include illustrative examples of various aspects and implementations, and provide an overview or framework for understanding the nature and character of the claimed aspects and implementations. The drawings provide illustration and a further understanding of the various aspects and implementations, and are incorporated in and constitute a part of this specification. It will be readily appreciated that features described in the context of one aspect of the invention can be combined with other aspects. Aspects can be implemented in any convenient form, for example, by appropriate computer programs, which may be carried on appropriate carrier media (computer readable media), which may be tangible carrier media (e.g., disks) or intangible carrier media (e.g., communications signals). Aspects may also be implemented using suitable apparatus, which may take the form of programmable computers running computer programs arranged to implement the aspect.

### BRIEF DESCRIPTION OF THE DRAWINGS

The foregoing and other objects, aspects, features, and advantages of the disclosure will become more apparent and better understood by referring to the following description taken in conjunction with the accompanying drawings, in which:

FIG. 1 depicts an example system for performing dynamic motion-resolved reconstruction from continuously acquired radial MRI data, in accordance with one or more implementations;

FIGS. 2A and 2B show an example of a feature and the feature having a plurality of radial samples thereof, in accordance with one or more implementations;

FIGS. 3A, 3B, and 3C show classified images of the features; FIG. 3D shows an average of the images of 3A, 3B, and 3C, in accordance with one or more implementations;

Referring to FIG. 4, a motion signal map 400 is depicted, according to some embodiments.

FIG. 5 shows a flow diagram for constructing an image domain image from a plurality of source images, in accordance with one or more implementations;

FIG. 6 depict a system for performing the flow of FIG. 5, in accordance with one or more implementations;

FIG. 7 depict another system for performing the flow of FIG. 5, in accordance with one or more implementations;

FIG. 8 shows a flow diagram for generating an image domain image from, in accordance with an illustrative embodiment;

FIG. 9 is a block diagram of a server system and a client computer system in accordance with an illustrative embodiment.

### DETAILED DESCRIPTION

The present techniques can reconstruct accelerated 4D MRI data using time-space-coil convolutional networks without k-space data consistency to remove aliasing artifacts from acceleration and significantly reduce computation time relative to iterative reconstruction algorithms. The images can be resolved at a quality or time to inform diagnosis, treatment, planning, treatment evaluation or other care. It should be appreciated that various concepts introduced above and discussed in greater detail below may be implemented in any of numerous ways. The disclosed concepts are not limited to any particular manner of implementation. Examples of specific implementations and applications are provided primarily for illustrative purposes. For the purpose of better understanding the present disclosure, a brief overview of the sections of the detailed description may be helpful:
Section A describes systems and methods for performing dynamic motion-resolved image reconstruction.
Section B describes a network environment and computing environment which may be useful for practicing various embodiments described herein.
Section C describes an example embodiment, using the systems and methods herein to generate a series of image space representations of an anatomical feature displaced by a motion signal during a data acquisition time.

### A. Fast Motion-Resolved MRI Reconstruction Using Space-Time-Coil Convolutional Networks Without k-space Data Consistency (Movienet)

Medical images can include a plurality of captured data which is combined to form high resolution images. For example, a magnetic resonance imaging (MRI) machine can capture data in a frequency transform domain, such as k-space. The MRI machine can include a plurality of coils such that n k-space datasets can be generated for each Euclidean space image. Further, some motion can be of interest for some patients (e.g., swallowing, or a heartbeat), or can interfere with an area of interest (e.g., a heartbeat can interfere with a liver scan by displacing the liver). Thus, it may be advantageous to capture motion data and combine data from the plurality of coils to resolve an image, containing information from a plurality of datasets. However, transforms between the k-space and the Euclidean domain can be computationally expensive, and particularly, so for resolving images from a plurality of k-space images that results from a plurality of coils or a plurality of motion states, wherein coherency between the images is maintained.

The computational expense can increase an energy use to resolve an image, as well as increase a time for resolving the image. For example, a patient may need to wait a substantial period of time for an image to be resolved so that a provider can offer a diagnosis, treatment evaluation or other care following an MRI, or may need to schedule a later appointment. For some issues requiring immediate response (such as when a medical procedure is delayed until results are available), the processing time may delay care, or result in a suboptimal decision based on unavailable data, or relatively low-information data.

As noted above, the time to resolve certain k-space data to Euclidean space images can be greater than desired. Thus, an acceleration may be desirable. Detecting certain features of an image based on the k-space data, and individually resolving each of the k-space data sets can be less computationally involved. However, the various Euclidean space images may not, individually, contain adequate information or clarity, in at least some implementations. Thus, it may be advantageous for a system to receive the individually resolved images in the Euclidean domain, along with the data extracted from the k-space data. Such a system may resolve the plurality of images to at least one higher quality image, based on Euclidean space information. For example, the Euclidean space information can be computationally less expensive, or can be more admitting of parallel processing. Thus, the systems and methods herein disclosed may reduce an energy usage, mean time to diagnosis, improve image quality, and otherwise advance the state of the art of therapeutic and/or diagnostic imaging, such as MRI imaging.

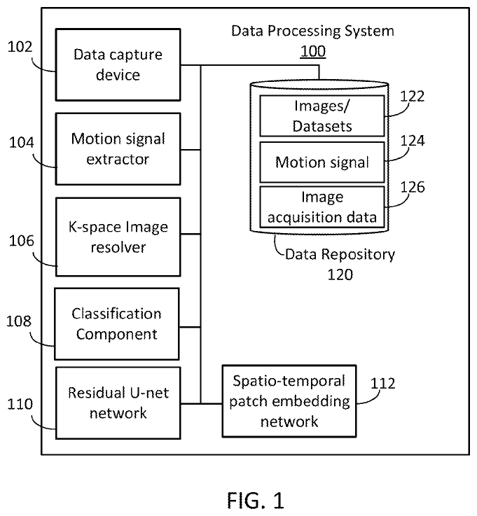

Referring now to FIG. 1, depicted is block diagram of an example data processing system 100, in accordance with one or more implementations. The data processing system 100 can include at least one data capture device 102. The data processing system 100 can include at least one motion signal extractor 104. The data processing system 100 can include at least one k-space image resolver 106. The data processing system 100 can include at least one classification component 108. The data processing system 100 can include at least one residual U-net network 110. The data processing system 100 can include at least one spatiotemporal patch embedding network 112. The data processing system 100 can include at least one data repository 120.

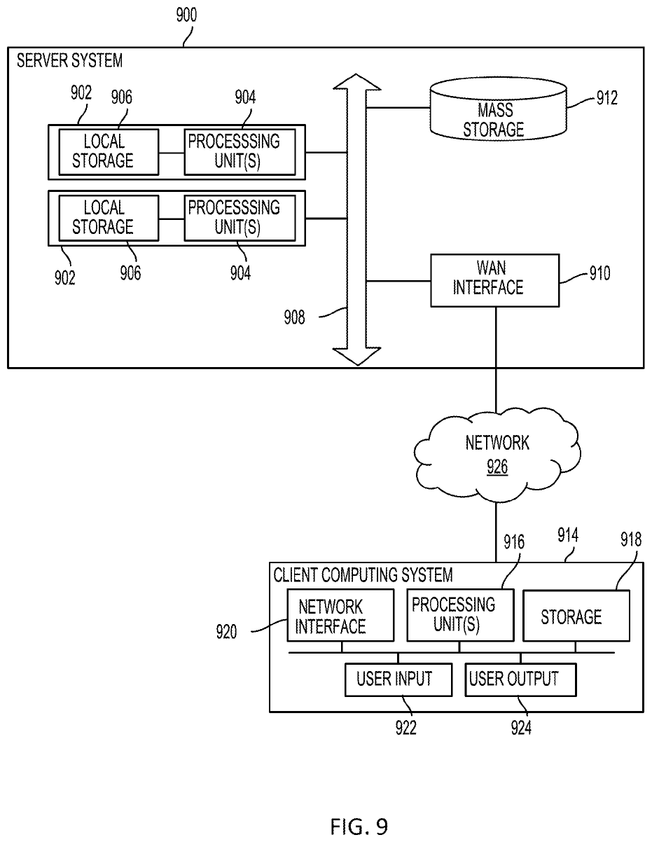

The data capture device 102, motion signal extractor 104, k-space image resolver 106, classification component 108, residual U-net network 110, or spatiotemporal patch embedding network 112 can each include a processing unit or other logic device such as programmable logic array engine, or module configured to communicate with the data repository 120 or database. The data capture device 102, motion signal extractor 104, k-space image resolver 106, classification component 108, residual U-net network 110, or spatiotemporal patch embedding network 112 can be separate components, a single component, or part of the data processing system 100. The data processing system 100 can include hardware elements, such as one or more processors, logic devices, or circuits. For example, the data processing system 100 can include one or more components, structures or functionality of a computing device depicted in FIG. 9.

The data repository 120 can include one or more local or distributed databases, and can include a database management system. The data repository 120 can include computer data storage or memory and can store a plurality of medical images 122. The images 122 can include an MRI image (e.g., slice thereof) such as an MRI image of an anatomical feature of interest such as a pathological feature (e.g., a lesion). The images 122 can comprise k-space data sets or Euclidean space images (e.g., derived from the k-space data sets). The data repository 120 can store a motion signal 124 or associated data. For example, the motion signal 124 can be a displacement associated with a cardiac or respiratory cycle which may be associated with one or a plurality of the medical images 122. The data repository 120 can store image acquisition data 126. For example, the image acquisition data 126 can include an acquisition time, acquisition coil, or other information associated with the acquisition of the data (e.g., the k-space data).

Still referring to FIG. 1, and in further detail, the data processing system 100 can include at least one data capture device 102 designed, constructed or operational to acquire data of an anatomical feature. For example, the data capture device 102 can capture k-space data for a three-dimensional anatomical feature. The data capture device can include a plurality of coils disposed radially around a feature of interest (e.g., an anatomical feature of interest). An electric field (e.g., an RF signal) can be passed through the feature of interest which can alternatively align the poles of portions thereof, such that upon a relaxation of the electric field (e.g., according to an electric field aligned with a spatial dimension) detectable energy can be emitted from the re-alignment (e.g., un-alignment) of the poles. For example, the energy can be measured in response to a gradient pulse and an RF (radio frequency) emission, and such a transfer function (e.g., measured response) can be mapped to the gradient or RF pulse (e.g., diagnostic or therapeutic information can be received in a k-space domain).

The data capture device 102 can capture a k-space data set, such as 2D data sets (also referred to as “slices,” herein) or 3D data sets. In some embodiments, the data capture device 102 can sample k-space data in alignment with a plurality of Euclidean space coordinates (e.g., Cartesian coordinates). For example, the data can be sampled according to an orthogonal frequency and gradient phase direction. The data capture device 102 cam pass such k-space data sets to the k-space image resolver 106. The data capture device 102 can maintain a Cartesian coordinate system. Such a coordinate system can maintain a constant phase and frequency directions which can localize movement. Such a coordinate system can sample a low frequency region of the k-space located near the origin thereof less than other methods disclosed herein, and peripheral regions with relatively high frequencies, which may emphasize high frequency data (e.g., detail) over low frequency data (e.g., contrast of major features).

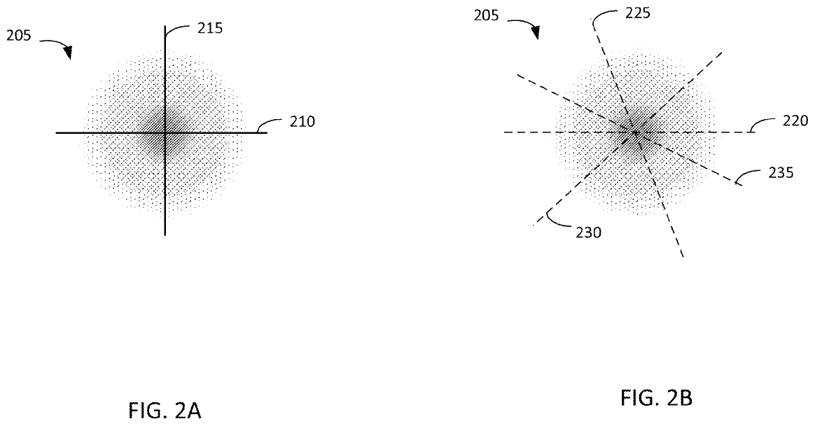

In some embodiments, the data capture device 102 can employ a non-Cartesian coordinate system. For example, the data capture device 102 can fill one or more radial portions of the k-space by various methods, such as filling the k-space associated with one or more rotating parallel lines. Such a radial system can sample the relatively information-dense center of the k-space more frequently that the Cartesian grids and the relatively information-sparse periphery of the k-space less frequently. Moreover, the changing angle of the data collection can disperse motion across the image. FIG. 2 discusses one radial method in further detail. The method is not intended to be limiting, indeed, the methods and systems disclosed herein can be performed according to data collected by various techniques.

The data processing system 100 can include at least one motion signal extractor 104 designed, constructed, or operational to define or identify a motion signal 124 from a plurality of the datasets captured by the data capture device 102. For example, motion signal extractor 104 can define or identify the motion signals 124 in k-space. In some embodiments, the motion signal extractor 104 can a define a period of a motion, such as a respiratory cycle which is controlled (e.g., coached) during data acquisition to maintain a regular and defined cadence. For example, the breathing can be responsive to a human machine interface communicatively coupled to the motion signal extractor 104 for provision to a patient (e.g., by audial or visual indication). In some embodiments, a patient can free-breath during the course of an examination, and thus a rate of respiration may be unknown. Further, a heartrate of a patient may not be readily controlled, and thus may require detection. Examples of respiratory and cardiac cycles are further discussed with regard to FIG. 4. In some embodiments, the motion signal extractor 104 can detect the motion signal 124 in Euclidean space.

In some embodiments, the motion signal extractor 104 can detect the motion signal in k-space. For example, the motion signal extractor 104 can detect a displacement of a center of a k-space line (e.g., relative to other k-space lines) and fit the displacements to predefined frequencies (e.g., according to a single dimensional Fourier transform). For example, a predefined frequency range can be defined, having a range of 0.1 Hz to 0.5 Hz, and another predefined frequency range can be defined having a range of 0.5 to 2.5 Hz. A peak detected frequency within the predefined ranges can be selected as the motion signal 124 of interest. The frequency ranges can be selected according to one or more patient attributes (e.g., to a cardiac or respiratory cycle), though such an embodiment is non-limiting. For example, the predefined frequency range can be associated with a nearby generator. In some embodiments, a plurality of frequencies can be associated with a feature. For example, a cardiac cycle can be disaggregated into a plurality of constituent cycles.

The data processing system 100 can include at least one k-space image resolver 106 designed, constructed, or operational to resolve a Euclidean space image from the k-space data. In some embodiments, the k-space image resolver 106 can resolve images by performing an inverse Fourier transform on k-space data sorted according to a Cartesian scheme. In some embodiments, the k-space image resolver 106 can associate a plurality of points disposed along a non-Cartesian scheme, such as a radial sampling of the k-space data to Cartesian coordinates and thereafter transforming the Cartesian coordinates to Euclidean space.

In some embodiments, the k-space image resolver 106 can manipulate a plurality of individual slices of k-space data to generate a plurality of two-dimensional images which can then be combined (e.g., stitched) to form a three-dimensional image. For example, the k-space image resolver 106 can combine two dimensional images captured by the data capture device 102. In some embodiments, the k-space image resolver 106 can receive three-dimensional k-space data from the data capture device 102. The k-space image resolver 106 can resolve images according to the three-dimensional data. For example, the k-space image resolver 106 can receive data sampled by the data capture device 102, according to a three-dimensional method (e.g., a three dimensional radial method, sometimes referred to as a “kooshball” method).

The k-space image resolver 106 can generate a three-dimensional image for each of a plurality of coils of the data capture device 102. In some embodiments, the k-space image resolver 106 can combine a plurality of three-dimensional images taken from a plurality of coils. For example, the k-space image resolver 106 can perform temporal compressed sensing reconstruction of a plurality of images to resolve a high-resolution image. For example, the k-space image resolver 106 can generate training data for either of the residual U-net network 110 or the spatio-temporal patch embedding network 112. The k-space image resolver 106 can receive a classification of a plurality of images from the classification component 108, which may increase a sparsity of device features (relative to a complete set of images comprising images in each of a plurality of the states of the motion signal 124). For example, the k-space image resolver 106 can resolve the function:

$${{‖ F \cdot C \cdot d - m ‖}_{2}^{2} + λ {‖ {S}_{1} \cdot d ‖}_{1} + {λ}_{2} ‖ {S}_{2} \cdot R \cdot d ‖}_{1}$$

Where F defines the non-uniform FFT (fast Fourier transform) or another Fourier transform, C represents a matrix sensitivity array, d is the 2D image series with a cardiac dimension and a respiratory dimension. S₁ is the sparsifying transform applied in the cardiac motion dimension with regularization parameter λ₁ and S₂ is the sparsifying transform applied along the extra respiratory-state dimension with regularization parameter λ₂. R is a reordering operator along the t_{R} dimension. In some embodiments, various terms may be substituted, replaced, omitted, or the like. For example, the R term can be omitted in embodiments having limited cardiac motion or which is otherwise accounted for.

The data processing system 100 can include at least one classification component 108 designed, constructed, or operational to classify the plurality of images to one or more portions of the motion signals 124. For example, the classification component 108 can classify one or more images (or portions thereof) according to a position. As will be further discussed with regard to FIG. 4, the classification component 108 can classify the images according to a position or a period. For example, an inhalation state which results in a similar displacement as an exhalation state can be classified as a same or different state. The classification component 108 can detect the presence of one or more outlier images (e.g., data which does not fit into a pre-defined or dynamically scaled class or state). The classification component 108 can pre-define the classes, or can align the classes to the image data or k-space data. In some embodiments, the classification component 108 can dynamically define the states to maintain an equal number of data sets in each class, a minimum number of data sets per state, a maximum number of data sets per state, a minimum variance between states, or otherwise establish or adjust thresholds associated with one or more states to disperse the available data between the states.

In some embodiments, the classification component 108 can classify Euclidean space images or k-space data sets according to a plurality of motion signals. For example, the cardiac rhythm and the respiratory rhythm can be classified along a two-dimensional space (e.g., defined by the displacement of the cardiac and respiratory cycle). In some embodiments, the classification component 108 can include user controlled or automatic detection of a desired state or defining cycle. For example, an MRI of a meniscus may have an associated displacement associated with a patient breathing, but little or no discernable relationship with a cardiac cycle. The classification component 108 can thus exclude the cardiac cycle, which may be responsive to a failure to detect a cardiac cycle exceeding a threshold, or an operator entry (e.g., manually deselecting a cardiac motion signal or selecting a knee MRI).

The data processing system 100 can include at least one residual U-net network 110 designed, constructed or operational to reconstruct a high-quality image from a plurality of three dimensional k-space images. The residual U-net network 110 can receive a training set of images from the k-space image resolver 106 which combine the 3D (or 2D) images from the plurality of coils and the plurality of motion signals states to a single processed image or a series of sorted processed images for each of the respective motion states. Subsequent to the training, the residual U-net network 110 can further receive a plurality of 3D images from the image resolver. For example, the residual U-net network 110 can receive a plurality of 3D images associated with image acquisition data 126 such as a coil associated with the image collection (e.g., RF coils to transmit or receive the data). The image acquisition data 126 can further include processed information such as a state of the motion signal 124 of an image, as classified by the classification component 108. In some embodiments, the U-net network 110 can operate on a plurality of two-dimensional planes of the three dimensional images. For example, the residual U-net network 110 can generate 256 two dimensional images having as resolution of 256×256 from a 3D image having a resolution of 256×256×256.

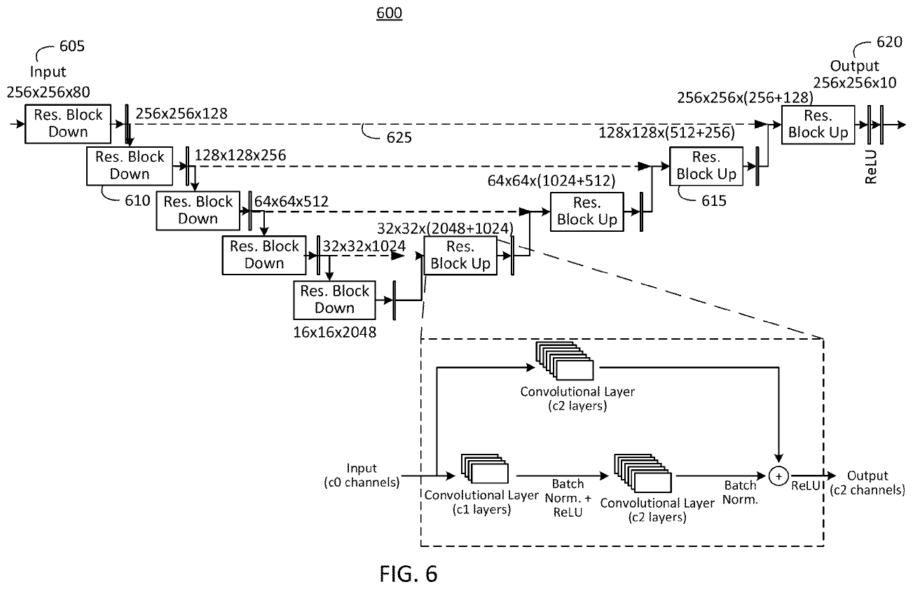

The U-net network 110 can concatenate the state of the motion signal 124 and the coils. For example, the U-net network 110 can receive the image data from a data capture device having eight coils, and a classification component classifying the images in ten motion signal states. The concatenated coils and motion signal 124 states can be disposed in the color dimension. Continuing the above example, each two-dimensional image can thus have a resolution of 256×256×80 (where 8 coils×10 motion signal states yields the resolution of 80). The U-net network 110 can process the images as is further described with respect to FIG. 6. For example, the U-net network 110 can down sample/encode the data, and thereafter up-sample/decode the image. In some embodiments, further networks such as a Mask-R CNN network, Feature Pyramid Network, and the like can be employed to perform, supplement, or validate the operation of the U-net network 110. In some embodiments, the residual U-net network 110 can be omitted, such as an embodiment exclusively employing the spatiotemporal patch embedding network 112.

The data processing system 100 can include at least one spatiotemporal patch embedding network 112 designed, constructed, or operational to reconstruct a high-quality image from the plurality of three dimensional images. The spatiotemporal patch embedding network 112 can receive the training data discussed with regard to the U-net network 110, and train the spatiotemporal patch embedding network 112. The loss function to train the spatiotemporal patch embedding network 112 can include a perceptual loss. The training of the model of the spatiotemporal patch embedding network 112 can be defined by or include a perceptual loss function. For example, the perceptual loss function can be another CNN, such as VGG (visual geometry group) 16 or VGG 19. The perceptual component of the loss function can be defined according to a formula:

$${L}_{perceptual} = {\sum }_{j} = {‖ {ϕ}_{j} ( \hat{y} ) - {ϕ}_{j} ( y ) ‖}_{2} + {λ}_{p} {‖ {ϕ}_{j} ( \hat{y} ) - {ϕ}_{j} ( y ) ‖}_{1}$$

The perceptual loss function can be combined with a pixelwise L₁ or L₂, which may mitigate or remove artifacts generated or tolerated by the perceptual loss function, such as high frequency artifacts. In some embodiments, the loss function can be applied to train a model on an image basis or slice basis. In some embodiments, the loss function can be applied to a plurality of models, such as based on patches of the image or images slices. The patches can be overlapping or non-overlapping. The spatiotemporal patch embedding network 112 can mix adjoining patches. For example, the spatiotemporal patch embedding network 112 can receive training from adjacent Euclidean space images (e.g., between the x, y, and z planes, or adjacent portions thereof), or image acquisition data dimensions (e.g., adjacent motion signal 124 states or coils). The adjacency of motion signal states or coils can be based on various criteria of adjacency including temporal, spatial, or associational. For example, a respiratory cycle can include spatially adjacent but temporally non-adjacent states (e.g., the middle of an exhale or inhale); non-adjacent coils can have a close association which are not spatially adjacent (e.g., offset 180°).

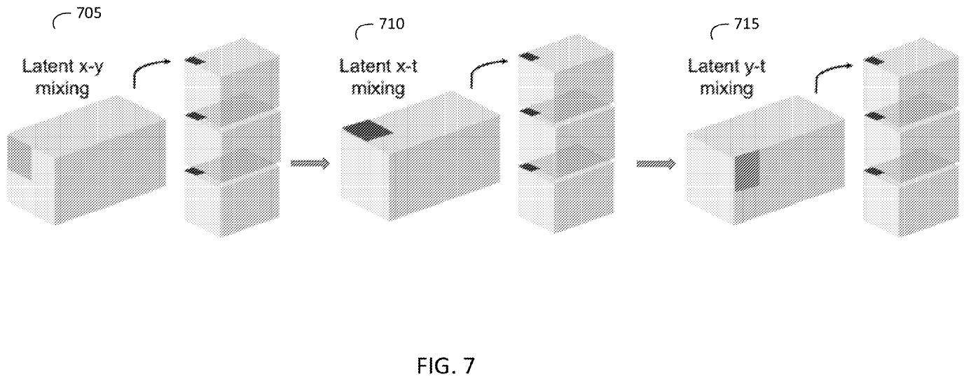

The spatiotemporal patch embedding network 112 can receive a plurality of images (e.g., images having associated image acquisition data 126 such as coil and motion states) to generate one or more processed images therefrom (e.g., can generate a plurality of high resolution or high contrast images therefrom). The spatiotemporal patch embedding network 112 can define patches across coordinates in the Euclidean space associated with a three dimensional image, as well as across the image acquisition data 126, including the motion signal 124 state or the coils associated with the image from the data capture device, which may be considered as five dimensions, or concatenated to four dimensions (as for the U-net residual network 110). The spatiotemporal patch embedding network 112 can thereafter mix the various dimensions, to train (or generate) images, as is further discussed with regards to FIG. 7. In some embodiments, the spatiotemporal patch embedding network 112 can be omitted, such as an embodiment exclusively employing the residual U-net network 110. In some embodiments, both networks can be employed such as to direct particular images to one or the other network based on operator selection, data content, or parallel resolution which can be compared (e.g., by the data processing system or an operator) for sharpness, contrast, information density, or the like.

Referring to FIG. 2A, an example of k-space 205 is depicted. The k-space 205 is depicted with relation to a coordinate system including an x-axis 210 separating a positive and negative component. The positive and negative components can be symmetrical. For example, the positive and negative components can be used to correct for various measurement or calculation errors by interpolation therebetween. The x-axis 210 can define a frequency of x-component frequencies of the k-space or an image corresponding thereto.

The y-axis 215 defines a magnitude of the frequency of y-component frequencies of the k-space or an image corresponding thereto. As depicted, FIG. 2A includes a relatively high concentration of low frequencies data (e.g., smooth shapes having high contrast therebetween) and a relatively low concentration of high frequency data (e.g., fine detail or rough/spiked patterns).

FIG. 2B depicts a series of radial lines disposed over the k-space, and can describe information sampled from the k-space. For example, a first radial line 220 samples a portion of the k space. A second radial line 225 samples a second portion of the k-space. The second radial line 225 can be offset from the first radial line a predefined distance, such as about 111.246°. A third radial line 230 and fourth radial line 235 can be offset a same or different distance. The angle can be selected to capture diverse components of the k-space 205. For example, a golden angle can be selected to avoid duplicate or near-duplicate (e.g., with 1 degree, 0.1 degrees, or so on) readings when a total number of samples is undefined. In some embodiments, a number of radial lines can be predefined, and the relative angles therebetween can be selected to equally divide the k-space 205. For example, for 180 pre-defined radial lines, the respective lines can be offset by 1°. The disclosure of one or more golden angle radial lines is not intended to be limiting. In some embodiments, one or more other methods (e.g., radial, spiral, linear, stack of stars, etc.) can be employed. The sampling can relate to points along the line selected for conversion to Euclidean space.

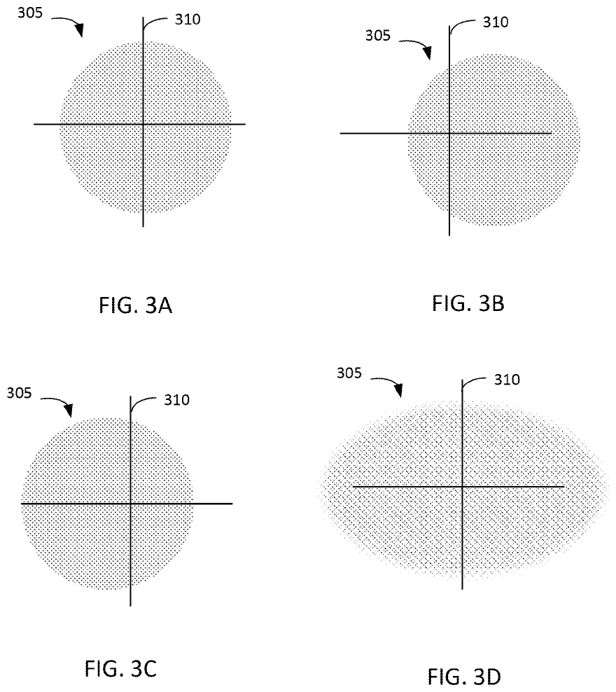

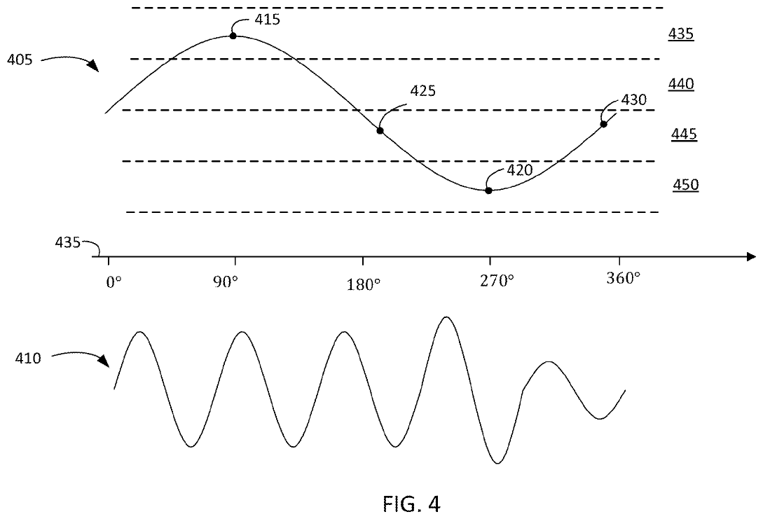

Referring to FIGS. 3A-3D, Euclidean space images of a feature 305 is depicted, according to a plurality of motion states. The feature is depicted as circular, merely for ease of depiction. One skilled in the art will understand that the feature can represent various anatomical organs, components, thereof, pathological features, and so on. For example, the feature can represent a liver or a lesion thereof. The motion can represent one or more motion signals 124 such as a cyclic motion signal (e.g., a cardiac cycle, or a respiratory cycle) or other time-variant behavior, such as swallowing, coughing, or the like.

At FIG. 3A, the feature 305 is depicted as aligned with an arbitrary axis 310. At FIG. 3B, the feature 305 is depicted as having an offset relative to the same arbitrary axis 310. At FIG. 3C, the feature 305 is depicted as having an opposite offset relative to the same arbitrary axis 310 as FIG. 3B. FIG. 3D represents a combinatorial depiction of the feature 305 relative to the arbitrary axis 310. FIGS. 3A-3C can depict single images, or combinations of a plurality of images of the feature 305 defined by a classification component 108 of the data processing system 100. For example, FIG. 3A can be a high quality image (e.g., high contrast image or high resolution image) generated by a combination of a plurality of images, such as by the residual u-net network 110, the spatiotemporal patch embedding network 112, or the k-space image resolver 106 (e.g., to train the residual u-net network 110 or the spatiotemporal patch embedding network 112). In some embodiments, the feature 305 may deform between states, which may further differentiate the variance between the images, and cause further information loss in FIG. 3D, or an analogue thereof. For example, the feature 305 can be a pericardial sack, and can deform according to a cardiac cycle.

Referring to FIG. 4, a motion signal map 400 is depicted, according to some embodiments. The motion signal map 400 includes a respiratory signal 405 and a cardiac signal 410. The respiratory signal 405 can include an inhalation peak 415, an exhalation peak 420, an inhalation midpoint 425 and an exhalation midpoint 430. The respiratory signal 405 can be generally cyclic, such that various measurements can be aligned along an axis 435 from 0° to 360°. For example, a plurality of measurements can be disposed in a frequency domain. For example, the center points of the radial lines of FIG. 2 can be transformed to generate a sinusoidal wave, such as the sinusoid of the respiratory signal 405. The respiratory signal can be subdivided into a plurality of states. For example, a first state 435 can include the inhalation peak 415. A second state 440 can be predefined to include additional data disposed there-within. A third state 445 can include an inhalation midpoint 425 and the exhalation midpoint 430. A fourth state 450 can include the exhalation midpoint 420. In some embodiments, the states can be disposed vertically over the sinusoid or other signal describing the respiratory motion (e.g., by angle, such that the inhalation midpoint 425 and exhalation midpoint 430 can be different states).

The cardiac signal 410 can be disposed along another axis (not depicted). For example, the cardiac signal 410 can be one or more higher frequency signals generated from the k-space 205. For example, the cardiac signal 410 can be resolved from k-space data 205 in combination with the respiratory signal 405 or additional signals. In some embodiments, the motion signal may not be non-regularly periodic. For example, a function (e.g., swallowing) can be repeated and aligned to an axis. As depicted, the cardiac signal 410 can include variance to the sinusoidal signal, such as frequency or amplitude variances. The variances can be included in the states, be defined as outliers, or can have additional states defined to capture the data thereof.

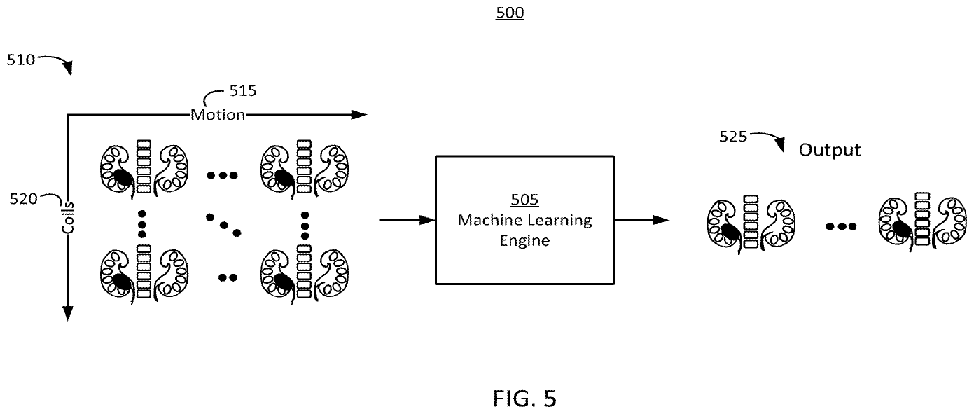

FIG. 5 depicts a flow diagram 500 for constructing a Euclidean space image from a plurality of Euclidean space input images 510, in accordance with one or more implementations. The flow diagram 500 can include a delivery of a plurality of input images 510 to a machine learning engine 505. The plurality of input images 510 can be disposed along a motion axis 515 (e.g., states of a motion signal 124). For example, each image can be associated with image acquisition data defining the position along an axis. The plurality of input images 510 can be associated with (e.g., can be generated responsive to k-state data 205 received by, or in response to a signal sent from the coil) various coils, along a coil axis 520. The output 525 of the machine learning engine 505 can contain a time-series of images, which can be generated from the input images 510. In some embodiments, each of the output images 525 can be derived from a single array of the input images 510. In some embodiments, the each of the output images 525 can be derived from multiple arrays of the input images 510 (e.g., based on the mixing between adjacent motion state images of the spatiotemporal patch embedding network 112).

FIG. 6 depicts an example representation 600 of a residual U-net network 110, in accordance with one or more implementations. The residual U-net network 110 receives an input image 605. For example, the input images 605 can be 256 pixels by 256 pixel image of a slice of a 3D image. A number of coils and time-series images can define a third dimension (sometimes referred to as a color dimension) of the input image 605 or set. In some embodiments, any of the dimensions of the images can be aligned to the residual U-net network 110 (e.g., by an alignment function performed by the residual U-net network 110 or another component of the data processing system 100). For example, the images can be adjusted centrally into the voxels of the residual U-net network 110, or aligned to a pre-defined edge thereof. In some embodiments, one or more padding datasets can be introduced, or the input image 605 can be otherwise resized to conform to a dimension of the U-net network 110.

The U-net network 110 includes a series of encoder blocks 610 and decoder blocks 615, and a series of lateral connections 625 therebetween. The encoder blocks 610 can pass pixel-wise data to the sequential encoder 610 or decoder 615 blocks; the lateral connections can pass information derived at various stages of the U-net network 110. For example, edge information can be derived from the input images having a higher resolution than the filtered images at other operations. Put differently, the image-wide information can be gathered in upper stages (e.g., disposed relatively near the input 605 or the output 620). The encoder blocks 610 and decoder blocks 615 can be symmetrical. For example, the encoder blocks 610 can perform a maximum pooling function (e.g., having a 2×2 pool and a stride of 2); the decoder block can perform a symmetrical up-sampling function.

FIG. 7 depicts a spatiotemporal patch embedding network 112, in accordance with one or more implementations. The spatiotemporal patch embedding network 112 can receive an input image 605. For example, the input image can be a same resolution, padding, and content as the input image of FIG. 6. In some embodiments, additional dimensions can be defined relative to the input image. For example, the input image 605 can be received as a 5-dimensional image including Cartesian dimensions, a dimension of the movement signal 124, and a dimension for the coils. Although X-Y-Z coordinates are described with respect to FIG. 7, the method can be performed with various coordinates for Euclidean space (e.g., polar coordinates or 2D coordinates).

At a first stage 705 of the spatiotemporal patch embedding network 112, pixels, or other pools of at least the X and Y coordinate are combined. For example, a convolutional layer can include a pixel or pool (e.g., the 2×2 pool of the U-net network 110) that can pass between the x and y dimensions, such that local information or context of a volumetric feature of the input image (e.g., a tumor or other lesion) having features disposed across the x and y dimensions can train or provide information for the spatiotemporal patch embedding network 112, or a portion thereof (e.g., a portion for a local patch). In some embodiments, other dimensions can be selected, such as the x and z dimensions or polar dimensions. Indeed, for the various stages disclosed herein, any two-dimensional projection of the 5 dimensional data can be selected. For example, the methods herein can include a two-dimensional projection including the coil dimensions.

At a second stage 710 of the spatiotemporal patch embedding network 112, the x dimension is mixed between adjacent dimensions of the motion signal 124. In some embodiments, the motion signal can include a linear set of images. In some embodiments, the motion signal 124 can “wrap around” such that all motion signals are adjacent to two other periodic signals, which can increase a number of relationships between periodic or repeating motion signals. At a third stage 715 of the spatiotemporal patch embedding network 112, y-t mixing is performed similarly to the x-y mixing of the second stage 710. The sequence of the stages disclosed herein is not intended to be limiting. Indeed, the sequences can be alternated or performed in various sequences (e.g., alternatively for separate embodiments, or sequentially in an embodiment). Advantageously, the mixing of the two-dimensional images can increase specificity of information to a patch of the 5D image, or reduce memory bandwidth required to convolve across the data sets.

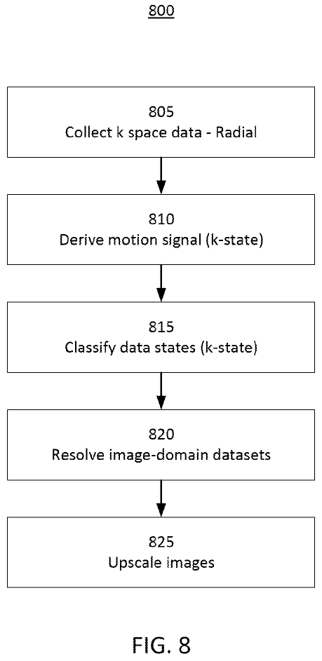

FIG. 8 depicts a method of generating images, according to some embodiments. The method 800 can be performed by one or more components or systems described in FIG. 1, 9, or throughout this disclosure. In brief summary, at operation 805, k-space data is collected. At operation 810, a motion signal 124 is derived from the k-space data. The data is classified according to the motion signal 124 at operation 815. The k-space data is resolved into image-domain datasets at operation 820. Resolved images are upscaled at operation 825.

At operation 805, the data capture device 102 collects the k-space data. For example, the data capture device can include the RF and gradient coils of an MRI machine transmitting or receiving information in a k-space domain to measure a magnitude of energy released from a slice or volume of tissue upon demagnetization. The k-space data can be collected according to a Cartesian coordinate system, or another coordinate system such as a radial coordinate system which can be resolved to generate a corresponding Cartesian coordinate. For example, a series of radial images can be taken, wherein each radial image reduces a maximum radially defined un-sampled area of the k-space.

At operation 810, the motion signal extractor 104 extracts one or more motion signals 124 from the k-space data. For example, the motion signal extractor 104 can receive or calculate a pre-defined bound of a motion signal 124 such as a cardiac signal. The motion signal extractor can evaluate a center-point of the radial arm of operation 805, and resolve the information therein to a corresponding signal in the image space (e.g., may perform an inverse Fourier transform).

At operation 815, the classification component 108 classifies the k-space data according to the motion signal 124. For example, a time-series of images can be classified according to states of the motion signal 124. In some embodiments, the states can be dynamically generated to sort a number of k-space data sets for each state. In some embodiments, state bounds can be predefined, such a where a signal is well defined, and a number or approximate number of k-space datasets can be predicted (e.g., a heart rate of a sedated patient).

At operation 820, the k-space data is resolved into Euclidean space data (e.g., an image). The k-space data can be resolved into images for each of a plurality of states of the motion signal 124 and coils. The images can be organized (e.g., sorted, tagged, or associated with metadata) according to associated coils or states of the motion signal 124. For example, the three-dimensional images can be disposed across a coil axis and a motion signal axis. The motion signal axis can also be referred to as a temporal axis or a motion axis, though the images thereof are not necessarily of a sequential temporal order. For example, for at least some motion signals 124, the beginning and ending of the motion signal 124 can be defined arbitrarily.

At operation 825, the resolved images are upscaled. For example, the images can be received by either or both of the Residual U-net network 110 or the Spatio-temporal patch embedding network 112. In some embodiments, the resolved images can be compared (e.g., pixel-wise, patch-wise, or image-wise) to select an image, or images associated with both systems can be presented. In some embodiments, one or both networks can be selected according to a nature of the image, image acquisition data 126 such as a reason for the image, a region of a body imaged, or other criteria, such as a manual selection. Upscaling can improve one or more components of image quality and does not require an increase in resolution, per se. Indeed, the methods and systems disclosed herein may, in at least some embodiments, improve an image quality based on a sharpness, contract, or any other characteristic that can be associated with a loss function.

### B. Computing and Network Environment

Various operations described herein can be implemented on computer systems. FIG. 9 shows a simplified block diagram of a representative server system 900, client computer system 914, and network 926 usable to implement certain embodiments of the present disclosure. In various embodiments, server system 900 or similar systems can implement services or servers described herein or portions thereof. Client computer system 914 or similar systems can implement clients described herein. The system 100 described herein can be similar to the server system 900. Server system 900 can have a modular design that incorporates a number of modules 902 (e.g., blades in a blade server embodiment); while two modules 902 are shown, any number can be provided. Each module 902 can include processing unit(s) 904 and local storage 906.

Processing unit(s) 904 can include a single processor, which can have one or more cores, or multiple processors. In some embodiments, processing unit(s) 904 can include a general-purpose primary processor as well as one or more special-purpose co-processors such as graphics processors, digital signal processors, or the like. In some embodiments, some or all processing units 904 can be implemented using customized circuits, such as application specific integrated circuits (ASICs) or field programmable gate arrays (FPGAs). In some embodiments, such integrated circuits execute instructions that are stored on the circuit itself. In other embodiments, processing unit(s) 904 can execute instructions stored in local storage 906. Any type of processors in any combination can be included in processing unit(s) 904.

Local storage 906 can include volatile storage media (e.g., DRAM, SRAM, SDRAM, or the like) and/or non-volatile storage media (e.g., magnetic or optical disk, flash memory, or the like). Storage media incorporated in local storage 906 can be fixed, removable or upgradeable as desired. Local storage 906 can be physically or logically divided into various subunits such as a system memory, a read-only memory (ROM), and a permanent storage device. The system memory can be a read-and-write memory device or a volatile read-and-write memory, such as dynamic random-access memory. The system memory can store some or all of the instructions and data that processing unit(s) 904 need at runtime. The ROM can store static data and instructions that are needed by processing unit(s) 904. The permanent storage device can be a non-volatile read-and-write memory device that can store instructions and data even when module 902 is powered down. The term “storage medium” as used herein includes any medium in which data can be stored indefinitely (subject to overwriting, electrical disturbance, power loss, or the like) and does not include carrier waves and transitory electronic signals propagating wirelessly or over wired connections.

In some embodiments, local storage 906 can store one or more software programs to be executed by processing unit(s) 904, such as an operating system and/or programs implementing various server functions such as functions of the system 100 of FIG. 1 or any other system described herein, or any other server(s) associated with system 100 or any other system described herein.

“Software” refers generally to sequences of instructions that, when executed by processing unit(s) 904 cause server system 900 (or portions thereof) to perform various operations, thus defining one or more specific machine embodiments that execute and perform the operations of the software programs. The instructions can be stored as firmware residing in read-only memory and/or program code stored in non-volatile storage media that can be read into volatile working memory for execution by processing unit(s) 904. Software can be implemented as a single program or a collection of separate programs or program modules that interact as desired. From local storage 906 (or non-local storage described below), processing unit(s) 904 can retrieve program instructions to execute and data to process in order to execute various operations described above.

In some server systems 900, multiple modules 902 can be interconnected via a bus or other interconnect 908, forming a local area network that supports communication between modules 902 and other components of server system 900. Interconnect 908 can be implemented using various technologies including server racks, hubs, routers, etc.

A wide area network (WAN) interface 910 can provide data communication capability between the local area network (interconnect 908) and the network 926, such as the Internet. Technologies can be used, including wired (e.g., Ethernet, IEEE 802.3 standards) and/or wireless technologies (e.g., Wi-Fi, IEEE 802.11 standards).

In some embodiments, local storage 906 is intended to provide working memory for processing unit(s) 904, providing fast access to programs and/or data to be processed while reducing traffic on interconnect 908. Storage for larger quantities of data can be provided on the local area network by one or more mass storage subsystems 912 that can be connected to interconnect 908. Mass storage subsystem 912 can be based on magnetic, optical, semiconductor, or other data storage media. Direct attached storage, storage area networks, network-attached storage, and the like can be used. Any data stores or other collections of data described herein as being produced, consumed, or maintained by a service or server can be stored in mass storage subsystem 912. In some embodiments, additional data storage resources may be accessible via WAN interface 910 (potentially with increased latency).

Server system 900 can operate in response to requests received via WAN interface 910. For example, one of modules 902 can implement a supervisory function and assign discrete tasks to other modules 902 in response to received requests. Work allocation techniques can be used. As requests are processed, results can be returned to the requester via WAN interface 910. Such operation can generally be automated. Further, in some embodiments, WAN interface 910 can connect multiple server systems 900 to each other, providing scalable systems capable of managing high volumes of activity. Other techniques for managing server systems and server farms (collections of server systems that cooperate) can be used, including dynamic resource allocation and reallocation.

Server system 900 can interact with various user-owned or user-operated devices via a wide-area network such as the Internet. An example of a user-operated device is shown in FIG. 9 as client computing system 914. Client computing system 914 can be implemented, for example, as a consumer device such as a smartphone, other mobile phone, tablet computer, wearable computing device (e.g., smart watch, eyeglasses), desktop computer, laptop computer, and so on.

For example, client computing system 914 can communicate via WAN interface 910. Client computing system 914 can include computer components such as processing unit(s) 916, storage device 918, network interface 920, user input device 922, and user output device 924. Client computing system 914 can be a computing device implemented in a variety of form factors, such as a desktop computer, laptop computer, tablet computer, smartphone, other mobile computing device, wearable computing device, or the like.

Processor 916 and storage device 918 can be similar to processing unit(s) 904 and local storage 906 described above. Suitable devices can be selected based on the demands to be placed on client computing system 914; for example, client computing system 914 can be implemented as a “thin” client with limited processing capability or as a high-powered computing device. Client computing system 914 can be provisioned with program code executable by processing unit(s) 916 to enable various interactions with server system 900.

Network interface 920 can provide a connection to the network 926, such as a wide area network (e.g., the Internet) to which WAN interface 910 of server system 900 is also connected. In various embodiments, network interface 920 can include a wired interface (e.g., Ethernet) and/or a wireless interface implementing various RF data communication standards such as Wi-Fi, Bluetooth, or cellular data network standards (e.g., 3G, 4G, LTE, etc.).

User input device 922 can include any device (or devices) via which a user can provide signals to client computing system 914; client computing system 914 can interpret the signals as indicative of particular user requests or information. In various embodiments, user input device 922 can include any or all of a keyboard, touch pad, touch screen, mouse or other pointing device, scroll wheel, click wheel, dial, button, switch, keypad, microphone, and so on.

User output device 924 can include any device via which client computing system 914 can provide information to a user. For example, user output device 924 can include a display to display images generated by or delivered to client computing system 914. The display can incorporate various image generation technologies, e.g., a liquid crystal display (LCD), light-emitting diode (LED) including organic light-emitting diodes (OLED), projection system, cathode ray tube (CRT), or the like, together with supporting electronics (e.g., digital-to-analog or analog-to-digital converters, signal processors, or the like). Some embodiments can include a device such as a touchscreen that functions as both input and output device. In some embodiments, other user output devices 924 can be provided in addition to or instead of a display. Examples include indicator lights, speakers, tactile “display” devices, printers, and so on.

Some embodiments include electronic components, such as microprocessors, storage and memory that store computer program instructions in a computer readable storage medium. Many of the features described in this specification can be implemented as processes that are specified as a set of program instructions encoded on a computer readable storage medium. When these program instructions are executed by one or more processing units, they cause the processing unit(s) to perform various operations indicated in the program instructions. Examples of program instructions or computer code include machine code, such as is produced by a compiler, and files including higher-level code that are executed by a computer, an electronic component, or a microprocessor using an interpreter. Through suitable programming, processing unit(s) 904 and 916 can provide various functionality for server system 900 and client computing system 914, including any of the functionality described herein as being performed by a server or client, or other functionality.

It will be appreciated that server system 900 and client computing system 914 are illustrative and that variations and modifications are possible. Computer systems used in connection with embodiments of the present disclosure can have other capabilities not specifically described here. Further, while server system 900 and client computing system 914 are described with reference to particular blocks, it is to be understood that these blocks are defined for convenience of description and are not intended to imply a particular physical arrangement of component parts. For instance, different blocks can be but need not be located in the same facility, in the same server rack, or on the same motherboard. Further, the blocks need not correspond to physically distinct components. Blocks can be configured to perform various operations, e.g., by programming a processor or providing appropriate control circuitry, and various blocks might or might not be reconfigurable depending on how the initial configuration is obtained. Embodiments of the present disclosure can be realized in a variety of apparatus including electronic devices implemented using any combination of circuitry and software.

### C. Example Embodiment

Motion-resolved 4D imaging (motion as the fourth dimension) is an important tool for radiation therapy of tumors affected by physiological motion, including respiratory motion, cardiac motion, peristaltic motion, swallowing motion and combinations. 4D imaging can be used to personalize treatment planning according to the motion range of the tumor and the surrounding organs with the goal to maximize radiation dose in the tumor and minimize radiation dose in the organs at risk. MRI presents several advantages over CT for 4D imaging, including superior soft tissue contrast to image organs at risk next to the tumor and the absence of ionizing radiation, which enables to scan for a longer time and obtain an improved characterization of motion. However, despite the extensive research on 4D MRI, there is no 4D MRI method currently available in clinical practice. One of the main reasons is the high data acquisition and computational burden to generate 4D MRI with sufficient spatial and temporal resolution.

eXtra-Dimensional Golden-angle RAdial Sparse Parallel MRI (XDGRASP) performs a continuous 3D golden-angle radial acquisition during motion and reconstructs a motion-resolved 4D image by sorting the acquired data into motion states and performing a temporal compressed sensing reconstruction. XD-GRASP enables 4D MRI with high spatial resolution (1-1.5 mm) and respiratory motion resolution (e.g., 10-20 states) within clinical acquisition times (5-6 min). However, the image reconstruction time can be long (10-30 minutes), which has prevented clinical implementation. There are several factors that increase reconstruction time, including (1) compressed sensing reconstruction can be iterative and requires several iterations to achieve high reconstruction performance, (2) data consistency in k-space can be performed in each iteration, which can involve computing two non-uniform fast Fourier transform (NUFFT). Moreover, the NUFFT can be applied in each coil and motion state, which can increase computation time.

Deep learning using convolutional neural networks can be applied to reconstruct dynamic MRI data, such as for cardiac motion, which can include obtaining a fully-sampled reference for training. These networks can unroll the iterations of compressed sensing as layers of the network and since they are trained based on several datasets, the number of layers can be reduced with respect to the number of iterations. However, they still perform data consistency in each layer, in at least some embodiments, and thus reconstruction time is still significant, particularly for high-dimensional data sets such as 4D MRI.

The present example can include a convolutional neural network that exploits correlations in 5D image space (x, y, z, time, coil), without enforcing data consistency in k-space, to remove aliasing artifacts from accelerated data acquisition and reconstruct an unaliased motion-resolved 4D image (e.g., FIG. 5). The dimension “time” can refer to motion states. The ability to remove k-space data consistency can accelerate the image reconstruction process, offering shorter reconstruction times than unrolled-loop convolutional neural networks, which can use data consistency in each layer.

The present example can operate entirely in the image domain without enforcing data consistency in k-space to remove aliasing artifacts in the 5D input image and reconstruct a motion-resolved unaliased 4D image. Time in this example refers to the motion dimension.

The input 5D image (x-y-z-time-coil) can be obtained according to at least of methods and techniques of the XD-GRASP technique, such as before temporal compressed reconstruction to convert 3D k-space data into motion resolved 4D k-space data. The 3D k-space data can be, for example, stack-of-stars (radial ky-kx, Cartesian kz) or 3D radial (kooshball). The following steps can be performed: 1) the center of k-space in each radial line can be sorted as a matrix and principal component analysis can be used to estimate a respiratory motion signal, 2) amplitude binning can be used to divide the amplitude of the respiratory motion signal into motion states, and 3) 3D k-space data in each coil can be sorted into motion states, such as according to the amplitude of each radial line in the respiratory motion signal.

The present example can be employed with at least a U-net with residual learning units, or convolutional space-time-coil patch embedding (e.g., the residual U-net network 110 or the spatiotemporal patch embedding network 112). Both versions can use iterative temporal compressed sensing results as a reference to train the network.

The residual U-net network 110 can concatenate the coil and time dimensions to exploit spatial correlations along both the coil and time dimensions to separate image features from image artifacts. Skip connections (dashed lines of FIG. 6) can be used to transfer features at the different levels of down-sampling and up-sampling. K-space data consistency can be obviated which can reduce a number of computationally expensive NUFFT transforms in each time point and coil.

The network can use a smooth L₁-loss function given by ΣIₙ, for Iₙ=l . . . N, with N representing the batch size, and lₙ. defined as 0.5(xₙ−yₙ)², if abs(xₙ−yₙ)<1, and abs(xₙ−yₙ)−0.5 otherwise, for a given output xₙ and target yₙ. This loss function can combine some advantages of the L₁-loss function that may be less impacted by outliers of the L₂-loss function according to some implementations.

FIG. 7 shows space-time-coil patch embedding and mixing, where spatial features localized in small patches are mixed along the time and coil dimensions. Conceptually, the idea is related to applying compressed sensing or low-rank matrix completion on small patches rather than on the whole image. Some image features are localized, and they are shared among temporal points and coils. By operating on patches, fewer datasets may be required to train the network, since multiple patches can be extracted from a single dataset, according to some embodiments. Mixing can separate learning across or within patches, creating highly specialized, efficient networks that may employ fewer trainable parameters, according to some implementations. Combining 3D patch embedding with 2D cross-sectional mixing layers enables exploration of correlations in various planes. Additionally, mixing may reduce computational complexity of convolutional blocks, which may further minimize reconstruction time, according to some implementations.

This network can optimize a multi-space loss function that can span at least two landscapes: (1) a perceptual loss that reduces the semantic differences between the reconstruction and target volumes in abstract feature spaces, which can be implemented via forward propagation through and probing of a separate, pre-trained vision network, such as VGG-16, and (2) a low-level quantitative pixelwise L₁ loss in raw image space that aims to correct any high-frequency artifacts produced by perceptual loss.

While the disclosure has been described with respect to specific embodiments, one skilled in the art will recognize that numerous modifications are possible. Embodiments of the disclosure can be realized using a variety of computer systems and communication technologies including but not limited to specific examples described herein. Embodiments of the present disclosure can be realized using any combination of dedicated components and/or programmable processors and/or other programmable devices. The various processes described herein can be implemented on the same processor or different processors in any combination. Where components are described as being configured to perform certain operations, such configuration can be accomplished, e.g., by designing electronic circuits to perform the operation, by programming programmable electronic circuits (such as microprocessors) to perform the operation, or any combination thereof. Further, while the embodiments described above may make reference to specific hardware and software components, those skilled in the art will appreciate that different combinations of hardware and/or software components may also be used and that particular operations described as being implemented in hardware might also be implemented in software or vice versa.

Computer programs incorporating various features of the present disclosure may be encoded and stored on various computer readable storage media; suitable media include magnetic disk or tape, optical storage media such as compact disk (CD) or DVD (digital versatile disk), flash memory, and other non-transitory media. Computer readable media encoded with the program code may be packaged with a compatible electronic device, or the program code may be provided separately from electronic devices (e.g., via Internet download or as a separately packaged computer-readable storage medium).

Thus, although the disclosure has been described with respect to specific embodiments, it will be appreciated that the disclosure is intended to cover all modifications and equivalents within the scope of the following claims.

Aspects can be combined, and it will be readily appreciated that features described in the context of one aspect can be combined with other aspects. Aspects can be implemented in any convenient form. For example, by appropriate computer programs, which may be carried on appropriate carrier media (computer readable media), which may be tangible carrier media (e.g., disks) or intangible carrier media (e.g. communications signals). Aspects may also be implemented using a suitable apparatus, which can take the form of one or more programmable computers running computer programs arranged to implement the aspect. As used in the specification and in the claims, the singular form of ‘a’, ‘an’, and ‘the’ include plural referents unless the context clearly dictates otherwise. References to “or” may be construed as inclusive so that any terms described using “or” may indicate any of a single, more than one, and all of the described terms. In some instances, this disclosure recites “and/or” which can also refer to all of the described terms as well as a single instance of the described terms or another subset of the described terms, without limiting effect to other instances of “or.”

What is claimed is:

1. A system for reconstruction of motion-resolved magnetic resonance images, the system comprising:
one or more processors coupled to a non-transitory memory, the one or more processors configured to:
receive a plurality of k-space data sets;
detect a motion signal from the plurality of k-space data sets;
classify each of the plurality of k-space data sets according to a state of the motion signal;
resolve the plurality of the k-space data sets to a plurality of first images, each of the plurality of first Euclidean space images corresponding to one of the plurality of k-space data sets;
convey the plurality of first Euclidean space images and corresponding image acquisition data to an image reconstruction convolutional network; and
resolve, by the image reconstruction convolutional network, a second Euclidean space image, based on the plurality of first Euclidean space images.

2. The system of claim 1, wherein the k-space data sets are radially sampled.

3. The system of claim 1, wherein the k-space data sets are received from a magnetic resonance imaging (MRI) machine.

4. The system of claim 3, wherein the k-space data sets are generated for each of a plurality of coils of the MRI machine.

5. The system of claim 1, wherein:
the motion signal corresponds to a respiratory cycle; and
the states of the motion signal are defined according to an amplitude and/or a phase of the motion signal.

6. The system of claim 1, wherein the k-space data sets are three dimensional.

7. The system of claim 4, wherein the image reconstruction convolutional network is a residual U-net network.

8. The system of claim 4, wherein the image reconstruction convolutional network employs patch mixing between at least Euclidean, coil, and motion signal state axes.

9. A method for resolving dynamic magnetic resonance images, the method comprising:
receiving, by a data processing system, a plurality of k-space data sets;
detecting, by the data processing system, a motion signal from the plurality of k-space data sets;
classifying, by the data processing system, each of the plurality of k-space data sets according to a state of the motion signal;
resolving, by the data processing system, the plurality of the k-space data sets to a plurality of first images, each of the plurality of first images corresponding to one of the plurality of k-space data sets;
conveying, by the data processing system, the plurality of first images and corresponding image acquisition data to an image reconstruction convolutional network of the data processing system; and
resolving, by the image reconstruction convolutional network of the data processing system, a second image, based on the plurality of first images.

10. The method of claim 9, wherein the k-space data sets are radially sampled.

11. The method of claim 9, wherein the k-space data sets are received from a magnetic resonance imaging (MRI) machine.

12. The method of claim 9, wherein the k-space data sets are generated for each of a plurality of coils of the MRI machine.

13. The method of claim 9, wherein:
the k-space data sets are three dimensional;
the motion signal is one-dimensional;
the motion signal corresponds to a respiratory cycle; and
the states of the motion signal are defined according to an amplitude and/or a phase of the motion signal.

14. The method of claim 12, wherein the image reconstruction convolutional network is a residual U-net network.

15. The method of claim 12, wherein the image reconstruction convolutional network employs patch mixing between at least a plurality of Euclidean axes, a coil axis, and a motion signal state axis.

16. A computer-readable medium storing instructions that, when executed by a one or more processors, cause it to perform a process comprising:
receiving a plurality of k-space data sets;
detecting a motion signal from the plurality of k-space data sets;
classifying each of the plurality of k-space data sets according to a state of the motion signal;
resolving the plurality of the k-space data sets to a plurality of first images, each of the plurality of first images corresponding to one of the plurality of k-space data sets;
conveying the plurality of first images and corresponding image acquisition data to an image reconstruction convolutional network; and
resolving a second image, based on the plurality of first images.

17. The computer-readable medium of claim 16, wherein the k-space data sets are received from each of a plurality of coils of an MRI machine.

18. The computer-readable medium of claim 16, wherein:
the k-space data sets are three dimensional;
the motion signal is one-dimensional;
the motion signal corresponds to a respiratory cycle; and
the states of the motion signal are defined according to an amplitude and/or a phase of the motion signal.

19. The computer-readable medium of claim 16, wherein the image reconstruction convolutional network is a residual U-net network.

20. The computer-readable medium of claim 16, wherein the image reconstruction convolutional network employs patch mixing between at least a plurality of Euclidean axes, a coil axis, and a motion signal state axis.

21. The method of claim 12, comprising:
detecting a further motion signal;
wherein:
the k-space data sets are three dimensional;
at least one of the motion signal or the further motion signal corresponds to a respiratory cycle;
the states of the motion signal are defined according to an amplitude and/or a phase of the motion signal; and
classifying each of the plurality of k-space data sets is based on the motion signal and the further motion signal.
